(19) United States
(12) Patent Application Publication
(10) Pub. No.: US 2022/0071711 A1
(43) Pub. Date: Mar. 10, 2022

(54) DEVICES, SYSTEMS, AND METHODS FOR IDENTIFYING UNEXAMINED REGIONS DURING A MEDICAL PROCEDURE

(71) Applicant: KARL STORZ SE & Co. KG, Tuttlingen (DE)

(72) Inventor: Marios KYPEROUNTAS, Goleta, CA (US)

(21) Appl. No.: 17/012,974

(22) Filed: Sep. 4, 2020

(51) Int. Cl. A61B 34/20; G06T 17/00; G06T 7/00; G06T 7/593; A61B 34/30

(52) U.S. Cl. CPC A61B 34/20; G06T 17/00; G06T 7/0012; G06T 7/593; A61B 34/30; A61B 2090/3762; G06T 2207/20081; G06T 2207/10028; G06T 2207/10068; A61B 2034/301; G06T 2210/41

(57) ABSTRACT

At least one example embodiment is directed to a device including a memory including instructions, and a processor that executes the instructions to generate, during a medical procedure being performed by a clinician on an internal region of a patient, image data and depth data for the internal region. The instructions cause the processor to generate, during the medical procedure, a depth model of the internal region of the patient based on the depth data, determine that the image data of the medical procedure does not include image data for a section of the internal region based on the depth model, and cause one or more alerts to alert the clinician that the section of the internal region is unexamined.

### FIELD

The present disclosure is generally directed to devices, systems, and methods for identifying unexamined regions during a medical procedure.

### BACKGROUND

Modern medical procedures may be camera-assisted, with video and/or still images of the procedure being displayed in real time to assist a clinician performing a procedure with navigating the anatomy. In some cases, regions of the anatomy are left unexamined, for example, because a large region of the anatomy may look very similar. In other cases, disorientation due to lack of distinct features in the anatomy may lead to leaving part of the anatomy unexamined.

### BRIEF DESCRIPTION OF THE DRAWINGS

FIG. 1 illustrates a system according to at least one example embodiment;

FIG. 2 illustrates example structures for a medical instrument according to at least one example embodiment;

FIG. 3 illustrates a method according to at least one example embodiment;

FIG. 4 illustrates a method according to at least one example embodiment;

FIG. 5 illustrates a workflow for a medical procedure according to at least one example embodiment; and

FIG. 6 illustrates example output devices according to at least one example embodiment.

### SUMMARY

At least one example embodiment is directed to a device including a memory including instructions, and a processor that executes the instructions to generate, during a medical procedure being performed by a clinician on an internal region of a patient, image data and depth data for the internal region, generate, during the medical procedure, a depth model of the internal region of the patient based on the depth data, determine that the image data of the medical procedure does not include image data for a section of the internal region based on the depth model, and cause one or more alerts to alert the clinician that the section of the internal region is unexamined.

At least one example embodiment is directed to a system including a display, a medical instrument, and a device. The device includes a memory including instructions and a processor that executes the instructions to generate, during a medical procedure being performed by a clinician on an internal region of a patient, image data and depth data for the internal region, generate, during the medical procedure, a depth model of the internal region of the patient based on the depth data, determine that the image data of the medical procedure does not include image data for a section of the internal region based on the depth model, cause one or more alerts to alert the clinician that the section of the internal region is unexamined.

At least one example embodiment is directed to a method including generating, during a medical procedure being performed by a clinician on an internal region of a patient, image data and depth data for the internal region, generating, during the medical procedure, a depth model of the internal region of the patient based on the depth data, determining that the image data does not include image data for a section of the internal region based on the depth model, and causing one or more alerts to alert the clinician that the section of the internal region is unexamined.

### DETAILED DESCRIPTION

Endoscopes and other medical instruments for imaging an anatomy have a limited field-of-view and require the user to manipulate the endoscope to image a larger-field-of-view area within the anatomy. In some cases, regions of the anatomy are left unexamined, for example, because regions of the anatomy look similar to one another. Disorientation due to lack of distinct features in the anatomy is also another example that could lead to leaving part of the anatomy unexamined.

Inventive concepts relate to anatomic imaging systems and to diagnostic procedures where it is important to ensure that a targeted region of the anatomy is fully examined. For example, inventive concepts are directed to a system to assist with visual examination of anatomy in endoscopic or other medical procedure by identifying and indicating non-examined regions. The system can be used to generate information about how much of the anatomy was examined or imaged by an imaging sensor, as well as provide information (e.g., location, shape, size, etc.) about the regions of interest that were not examined. For example, the system could assist a surgeon or clinician to ensure that all of the anatomy that was intended to be examined for abnormalities was actually examined. The system is useful for procedures such as colonoscopies, bronchoscopies, laryngoscopies, etc.

In general, inventive concepts include generating depth data and visual imaging data and combining them (using known alignment and/or mapping operations) to create visualizations and to trigger alerts that can be used to indicate the parts of the anatomy that have and have not been imaged by the imaging system. The visualizations and alerts provide information (true or relative location, area, shape, etc.) for regions that have not been yet examined, or imaged, to help mitigate the risk of leaving regions that were meant to be examined, unexamined.

By graphing/building a 3D depth model, regions within the anatomy can be identified that have not been imaged in color by an imaging sensor. For example, discontinuities (e.g., missing data) in the 3D depth model itself can indicate that the region around the discontinuity has not been imaged by the color image sensor.

In at least one example embodiment, generic 3D models can be pre-generated (e.g., from depth data taken from other patients) and used to select the general region of the anatomy that should be examined. The alerts can then indicate if the selected regions were fully examined or not, as well as provide a measure and/or indicate of how much of the overall region was left unexamined, how many regions were left unexamined (blind-spots), and where those missing regions are located.

Even in the absence of a pre-generated generic 3D model, the user or clinician can have the ability to indicate what the general region of interest is, and once the endoscope reaches that general region of interest, to initiate generating the needed information by the mapping and measurement system of inventive concepts. The user can also have the ability to indicate when the endoscope reaches the end of the general region of interest, to then enable generation of additional metrics that can be used to indicate if the region of interest was fully examined, how much of it was examined, how many sub-regions were not examined (blind-spots), etc.

An additional feature of inventive concepts can help the user navigate the 3D space to get to regions that have not been yet examined, or were missed. This may be done by adding graphics to a display monitor (arrows), audio instructions (e.g., ‘keep going forward’, ‘unexamined region is on the left side’, etc.).

For medical endoscopy, relying only on 2D image data to determine if a region within the anatomy was fully examined is less reliable than also using depth data. For example, using 2D visual data is susceptible to reflections, over-exposure, smoke, etc. However, depth data is useful for mapping the anatomy because there are very few or no flat surfaces within the human anatomy that exceed the depth camera's field of view, which reduces or eliminates cases where depth data would not be able to reliably determine if a region was fully examined.

Accordingly, inventive concepts relate to systems, methods, and/or devices that generate visual imaging data as well as depth data concurrently or in a time-aligned manner, align the visual imaging data with the depth data and/or map one set of data to the other, generate a 3D model of the scene using the depth data, identify discontinuities (missing data) in the 3D model as the parts of the model in which no depth data exists, and infer that these parts of the scene that were not imaged by the imaging sensor (i.e., identify the regions where image/visual data is missing). Inventive concepts may create visualizations, alerts, measurements, etc. to provide information to the user about the regions that were and were not imaged, their location, their shape, the number of missed regions, etc.

Additionally, inventive concepts may use the depth and image/visual data to create a 3D reproduction or composite 3D model of the visual content of the scene by, for example, projecting or overlaying 2D visual or image data onto the 3D depth model using the corresponding depth data information. At least one example embodiment provides the user with the option to interactively rotate the 3D composite and/or depth model. Depth map representations for the depth model can be created using depth data and, optionally, image/visual data. As an example, four corresponding depth map illustrations with views from north, south, east, west can be generated for a specific scene of interest, with all four views derived from a single 3D depth model. As noted above, a system according to example embodiments identifies regions in the 3D model where data is missing to identify parts of the anatomy that have not been imaged or examined.

In at least one example embodiment, the current position of the endoscope or other instrument having the depth and/or imaging cameras can be indicated on the 3D depth model in order to help the user navigate to the part of the anatomy that has not been imaged (i.e., a ‘you are here’ feature). Optionally, the system can calculate and report metrics to the user that describe what region of the anatomy has not been imaged. For example, this can be based on multiple gaps within the region that has been imaged (e.g., number of sub-regions that have not been imaged, size of each sub-region, shortest distance between sub-regions, etc.). These metrics may then be used to generate alerts for the user, for example, audio and/or visual alerts that a certain region is unexamined.

In at least one example embodiment, the system uses the information about the missed regions to produce visualizations on a main or secondary live-view monitor, which may help the user navigate to the region(s) of the anatomy that have not been imaged/examined. For example, graphics, such as arrows, can be overlaid over the live video data and used to point the direction (e.g., arrow direction) and distance (e.g., arrow length or arrow color) of the region that has not been examined. Here, the user may have the option to select one of the regions that was missed/not-examined and direct the system to help the user navigate to that specific region. In at least one example embodiment, a pre-generated 3D model (e.g., generic model) of the anatomy can be used to allow the user to indicate the region(s) of the anatomy that would need to be examined before the surgical procedure takes place. Indications, visualizations, and/or alerts could then be generated based on the difference between the region that was intended to be examined and the region that was actually examined.

In at least one example embodiment, the system employs neural network or other machine learning algorithm to learn (using multiple manually performed procedures) the regions that should be examined in a specific type of procedure. This may help determine if any region that has not been imaged/examined should or should not be examined. The system may generate alerts/notifications to the user whenever an unexamined region is determined to be a region that should be examined.

In at least one example embodiment, a robotic arm can be included, for example, in the endoscopic system of devices, that can use the 3D depth map information and guide/navigate the endoscope or other device with cameras to the regions-of-interest that have not yet been imaged. For example, with the robotic arm, the user can select the region of interest that the system should navigate to and an algorithm that uses machine learning can be pre-trained and used to execute this navigation automatically, without user intervention or with minimal user intervention. The system may employ a second machine learning algorithm to create and recommend the most efficient navigation path so that all regions of interest that have not yet been imaged can be imaged in the shortest amount of time possible. The user can then approve this path and the robotic arm will automatically navigate/move the endoscope or other device with a camera along this path. Otherwise, the user can override the recommendation for the most efficient path and select the region of interest to which the robotic arm should navigate.

As noted above, depth and the image/visual information can be generated by using an imaging sensor that concurrently captures both depth and visual imaging data. This is a useful approach that simplifies aligning the two types of data, or the mapping from one type of data to the other. An example of this type of sensor is the AR0430 CMOS sensor. In at least one example embodiment, the system uses a first depth sensor (e.g., LIDAR) to gather depth data and a second imaging sensor to generate the image data. The image data and depth data are then combined by either aligning them or inferring a mapping from one set of data to the other. This enables the system to infer what image data correspond to the captured depth data, and, optionally, to project the visual data onto the 3D depth model. In at least one other example embodiment, the system uses two imaging sensors in a stereoscopic configuration to generate stereo capture of visual/image data of the scene, which the system can use to then infer the depth information from the stereo image data.

As noted above, example embodiments are able to identify the regions of an anatomy that have not been imaged, or examined. This is accomplished by creating the 3D (depth) model and identify discontinuities or missing depth information in the model (i.e., identify regions of the 3D model where no depth data exists). Optionally, based on the aforementioned discontinuities in the depth information, the system can infer what image/visual information should also be missing, for example, by producing visualizations on a display that show the regions with missing data. In the event that depth data is less sparse than image data, the resolution of the depth sensor is considered in order to determine if image data is really missing.

In order to determine if a missed/unexamined region should be examined, the system may identify gaps within the region that the user is examining (e.g., occluded areas). Optionally, the system uses input from the user about the overall region of interest. The user input may be in the form of identifying regions of interest on a generic 3D depth model. In at least one example embodiment, the system uses a machine learning algorithm or deep neural networks to learn, over time and over multiple manually performed procedures, the regions that should be examined for each specific type of procedure. The system can then compare this information against the regions that are actually being examined by the user to determine if any region that has not been imaged/examined should or should not be examined.

In the case of employing a pre-generated 3D model of the anatomy, the system may determine a position of the endoscope or other camera device using a known direct or inferred mapping scheme to map the pre-generated 3D model to the 3D model being generated in real time. In this case, the endoscope may include additional sensors that provide current location information. These additional sensors may include but are not limited to magnetometers, gyroscopes, and/or accelerometers. The precision of the information from these sensors need not be exact when, for example, the user has indicated a general area of interest on the pre-generated 3D model. Even if the estimated location is not sufficiently accurate, the selected area in the pre-generated model can be expanded to ensure that the region of interest is still fully examined. Additionally or alternatively, specific features within the anatomy can be identified and used as points of interest to determine when the endoscope enters and exits the region of interest that was set using the pre-generated 3D model. These features enable the user to have some known indication/cues on where the region of interest should start and end (e.g., the beginning and end of a lumen). In addition to depth data, the 3D pre-generated model can also include reference image/visual data to assist with the feature matching operation that aligns the live depth model with the pre-generated depth model. Thus, both depth and image data could be used for the feature matching and mapping operations.

As noted above, the pre-generated 3D model may be taken from a number of other depth models, generated from previously executed medical procedures. However, example embodiments are not limited thereto, and alternative methods can be used to create the pre-generated 3D model. For example, the pre-generated model may be derived from different modalities, such as a computed tomography (CT) scan of the anatomy prior to the surgical procedure. In this case, the pre-generated 3D model may be more custom or specific to the patient if the CT scan is of the same patient. Here, data of the CT scan is correlated with the real-time depth data and/or image data of the medical procedure.

In view of the foregoing and the following description, it should be appreciated that example embodiments provide a system that assists with the visual examination of anatomy, for example, in endoscopic procedures and other procedures on internal anatomies. For example, a system according to an example embodiment identifies and indicates unexamined regions during a medical procedure, which may take the form of gaps in the overall examined region or gaps in pre-determined or pre-selected regions. The system may generate alerts, metrics, and/or visualizations to provide the user with information about the unexamined regions. For example, the system may provide navigating instructions for user to navigate to regions that have not been examined. A pre-generated 3D model may assist the user with specifying regions of interest. The system may use deep learning algorithm to learn the regions that should be examined for each specific surgical procedure and then compare the learned regions against the regions that are actually being imaged/examined to generate live alerts for the user whenever an area is missed. In at least one example embodiment, the system employs a robotic arm that holds an endoscope, a machine learning algorithm that controls the robotic arm, and another machine learning algorithm to identify and instruct the robotic arm to follow the most efficient/fastest path from the current position of the endoscope to the unexamined region of interest, or to and through a set of unexamined regions-of-interest. These and other advantages will be apparent in view of following description.

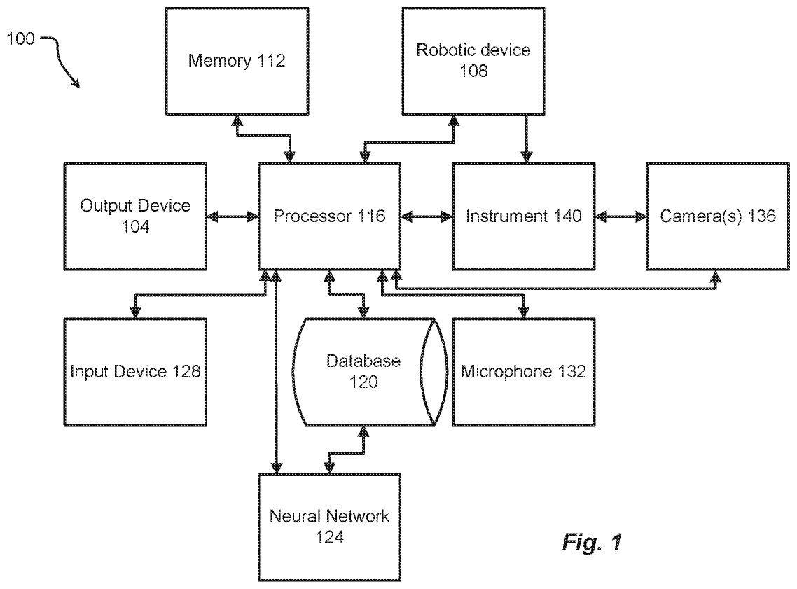

FIG. 1 illustrates a system 100 according to at least one example embodiment. The system 100 includes an output device 104, a robotic device 108, a memory 112, a processor 116, a database 120, a neural network 124, an input device 128, a microphone 132, camera(s) 136, and a medical instrument or tooling 140.

The output device 104 may include a display, such as a liquid crystal display (LCD), a light emitting diode (LED) display, or the like. The output device 104 may be a stand-alone display or a display integrated as part of another device, such as a smart phone, a laptop, a tablet, and/or the like. Although a single output device 104 is shown, the system 100 may include more output devices 104 according to system design.

The robotic device 108 includes known hardware and/or software capable of robotically assisting with a medical procedure within the system 100. For example, the robotic device 108 may be a robotic arm mechanically attached to an instrument 140 and in electrical communication with and controllable by the processor 116. The robotic device 108 may be an optional element of the system 100 that consumes or receives 3D depth map information and is able to guide/navigate the instrument 140 to regions of interest (ROIs) that have not yet been imaged. For example, where the robotic device 108 is a robotic arm, a user (e.g., a clinician) can select an ROI that the system should navigate to and the robotic arm, using an algorithm based on machine learning, can be pre-trained and used to execute this navigation automatically with little or no user involvement. Additionally, a second machine learning algorithm can be used to create and recommend the most efficient navigation path, so that all ROIs that have not yet been imaged can be imaged in the shortest amount of time possible. The user can then approve this path and the robotic arm will automatically navigate/move the instrument 140 along this path. Otherwise, the user can override the recommendation for the most efficient path and select the region of interest that the robotic arm should navigate to.

The memory 112 may be a computer readable medium including instructions that are executable by the processor 116. The memory 112 may include any type of computer memory device, and may be volatile or non-volatile in nature. In some embodiments, the memory 112 may include a plurality of different memory devices. Non-limiting examples of memory 112 include Random Access Memory (RAM), Read Only Memory (ROM), flash memory, Electronically-Erasable Programmable ROM (EEPROM), Dynamic RAM (DRAM), etc. The memory 112 may include instructions that enable the processor 120 to control the various elements of the system 100 and to store data, for example, into the database 120 and retrieve information from the database 120. The memory 112 may be local (e.g., integrated with) the processor 116 and/or separate from the processor 116.

The processor 116 may correspond to one or many computer processing devices. For instance, the processor 116 may be provided as a Field Programmable Gate Array (FPGA), an Application-Specific Integrated Circuit (ASIC), any other type of Integrated Circuit (IC) chip, a collection of IC chips, a microcontroller, a collection of microcontrollers, or the like. As a more specific example, the processor 116 may be provided as a microprocessor, Central Processing Unit (CPU), and/or Graphics Processing Unit (GPU), or plurality of microprocessors that are configured to execute the instructions sets stored in memory 112. The processor 116 enables various functions of the system 100 upon executing the instructions stored in memory 112.

The database 120 includes the same or similar structure as the memory 112 described above. In at least one example embodiment, the database 120 is included in a remote server and stores training data for training the neural network 124. The training data contained in the database 120 and used for training the neural network 124 is described in more detail below.

The neural network 124 may be an artificial neural network (ANN) implemented by one or more computer processing devices that are capable of performing functions associated with artificial intelligence (AI) and that have the same or similar structure of the processor 116 executing instructions on a memory having the same or similar structure as memory 112. For example, the neural network 124 uses machine learning or deep learning to improve the accuracy of a set of outputs based on sets of inputs (e.g., similar sets of inputs) over time. As such, the neural network 124 may utilize supervised learning, unsupervised learning, reinforcement learning, self-learning, and/or any other type of machine learning to produce a set of outputs based on a set of inputs. Roles of the neural network 124 are discussed in more detail below. Here, it should be appreciated that the database 120 and the neural network 124 may be implemented by a server or other computing device that is remote from the remaining elements of the system 100.

The input device 128 includes hardware and/or software that enables user input to the system 100. The input device 128 may include a keyboard, a mouse, a touch-sensitive pad, touch-sensitive buttons, a touch-sensitive portion of a display, mechanical buttons, switches, and/or other control elements for providing user input to the system 100 to enable user control over certain functions of the system 100.

The microphone 132 includes hardware and/or software for enabling detection and collection of audio signals within the system 100. For example, the microphone 132 enables collection of a clinician's voice, activation of medical tooling (e.g., the medical instrument 140), and/or other audio within an operating room.

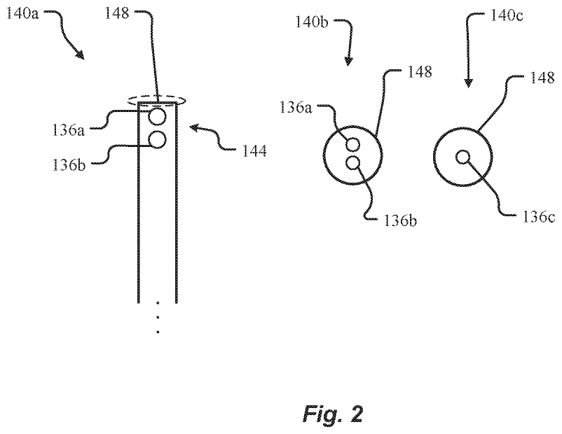

The camera(s) 136 includes hardware and/or software for enabling collection of video, images, and/or depth information of a medical procedure. In at least one example embodiment, the camera 136 captures video and/or still images of a medical procedure being performed on a body of patient. As is known in endoscopy, arthroscopy, and the like, the camera 136 may be designed to enter a body and take real-time video of the procedure to assist the clinician with performing the procedure and/or making diagnoses. In at least one other example embodiment, the camera 136 remains outside of the patient's body to take video of an external medical procedure. More cameras 136 may be included according to system design. For example, according to at least one example embodiment, the cameras 136 include a camera to capture image data (e.g., two-dimensional color images) and a camera to capture depth data to create a three-dimensional depth model. Details of the camera(s) 136 are discussed in more detail below with reference to FIG. 2.

The instrument or tooling 140 may be a medical instrument or medical tooling that is able to be controlled by the clinician and/or the robotic device 108 to assist with carrying out a medical procedure on a patient. The camera(s) 136 may be integrated with the instrument 140, for example, in the case of an endoscope. However, example embodiments are not limited thereto, and the instrument 140 may be separate from the camera 136 depending on the medical procedure. Although one instrument 140 is shown, additional instruments 140 may be present in the system 100 depending on the type of medical procedure. In addition, it should be appreciated that the instrument 140 may be for use on the exterior and/or in the interior of a patient's body.

Although FIG. 1 illustrates the various elements in the system 100 as being separate from one another, it should be appreciated that some or all of the elements may be integrated with each other if desired. For example, a single desktop or laptop computer may include the output device 104 (e.g., display), the memory 112, the processor 116, the input device 128, and the microphone 132. In another example, the neural network 124 may be included with the processor 116 so that AI operations are carried out locally instead of remotely.

It should be further appreciated that each element in the system 100 includes one or more communication interfaces that enable communication with other elements in the system 100. These communication interfaces include wired and/or wireless communication interfaces for exchanging data and control signals between one another. Examples of wired communication interfaces/connections include Ethernet connections, HDMI connections, connections that adhere to PCI/PCIe standards and SATA standards, and/or the like. Examples of wireless interfaces/connections include Wi-Fi connections, LTE connections, Bluetooth connections, NFC connections, and/or the like.

FIG. 2 illustrates example structures for medical instruments 140 including one or more cameras 136 mounted thereon according to at least one example embodiment. As noted above, a medical instrument 140 may include one or multiple cameras or sensors to collect image data for generating color images and/or depth data for generating depth images or depth models. FIG. 2 illustrates a first example structure of a medical instrument 140a that includes two cameras 136a and 136b arranged at one end 144 of the medical instrument 140a. The camera 136a may be an imaging camera with an image sensor for generating and providing image data and a depth camera 136b with a depth sensor for generating and providing depth data. The camera 136a may generate color images that include color information (e.g., RGB color information) while the camera 136b may generate depth images that do not include color information.

FIG. 2 illustrates another example structure of a medical instrument 140b where the cameras 136a and 136b are arranged on a tip or end surface 148 of the end 144 of the medical instrument 140b. In both example medical instruments 140a and 140b, the cameras 136a and 136b are arranged on the medical instrument to have overlapping fields of view. For example, the cameras 136a and 136b are aligned with one another in the vertical direction as shown, or in the horizontal direction if desired. In addition, the imaging camera 136a may swap positions with the depth camera 136b according to design preferences. An amount of the overlapping fields of view may be a design parameter set based on empirical evidence and/or preference.

It should be appreciated that the depth camera 136b includes hardware and/or software to enable distance or depth detection. The depth camera 136b may operate according to time-of-flight (TOF) principles. As such, the depth camera 136b includes a light source that emits the light (e.g., infrared (IR) light) which reflects off of an object and is then sensed by pixels of the depth sensor. For example, the depth camera 136b may operate according to direct TOF or indirect TOF principles. Devices operating according to direct TOF principles measure the actual time delay between emitted light and reflected light received from an object while devices operating according to indirect TOF principles measure phase differences between emitted light and reflected light received from the object, where the time delay is then calculated from phase differences. In any event, the time delay between emitting light from the light source and receiving reflected light at the sensor corresponds to a distance between a pixel of the depth sensor and the object. A specific example of the depth camera 136 is one that employs LIDAR. A depth model of the object can then be generated in accordance with known techniques.

FIG. 2 illustrates a third example structure of a medical instrument 140c that includes a combination camera 136b capable of capturing image data and depth data. The combination imaging and depth sensor 136c may be arranged on a tip 148 of the instrument 140c. The camera 136c may include depth pixels that provide the depth data and imaging pixels that provide the image data. As with medical instrument 140b, the medical instrument 140c further includes a light source to emit light (e.g., IR light) in order to enable collection of the depth data by the depth pixels. One example arrangement of imaging pixels and depth pixels includes the camera 136c having 2×2 arrays of pixels in Bayer filter configurations where one of the pixels in each 2×2 array that normally has a green color filter is replaced with a depth pixel that senses IR light. Each depth pixel may have a filter that passes IR light and blocks visible light. However, example embodiments are not limited thereto and other configurations for depth pixels and imaging pixels are possible depending on design preferences.

Although not explicitly shown for medical instrument 140a, it should be appreciated that a single camera 136c with image and depth sensing capabilities may be used on the end 144 of the instrument 140a instead of cameras 136a and 136b. Additionally, in at least one example embodiment, depth data may be derived from image data, for example, in a scenario where camera 136b in instrument 140a or instrument 140b is replaced with another camera 136a to form a stereoscopic camera from two cameras 136a that collect only image data. Depth data may be derived from a stereoscopic camera in accordance with known techniques, for example, by generating a disparity map from a first image from one camera 136a and a second image from the other camera 136a taken at a same instant in time.

Here, it should be appreciated that additional cameras 136a, 136b, and/or 136c may be included on the medical instrument 140 and in any arrangement according to design preferences. It should also be appreciated that various other sensors may be included on the medical instrument 140. Such other sensors include but are not limited to magnetometers, accelerometers, and/or the like, which can used to estimate the position and/or direction of orientation of the medical instrument 140.

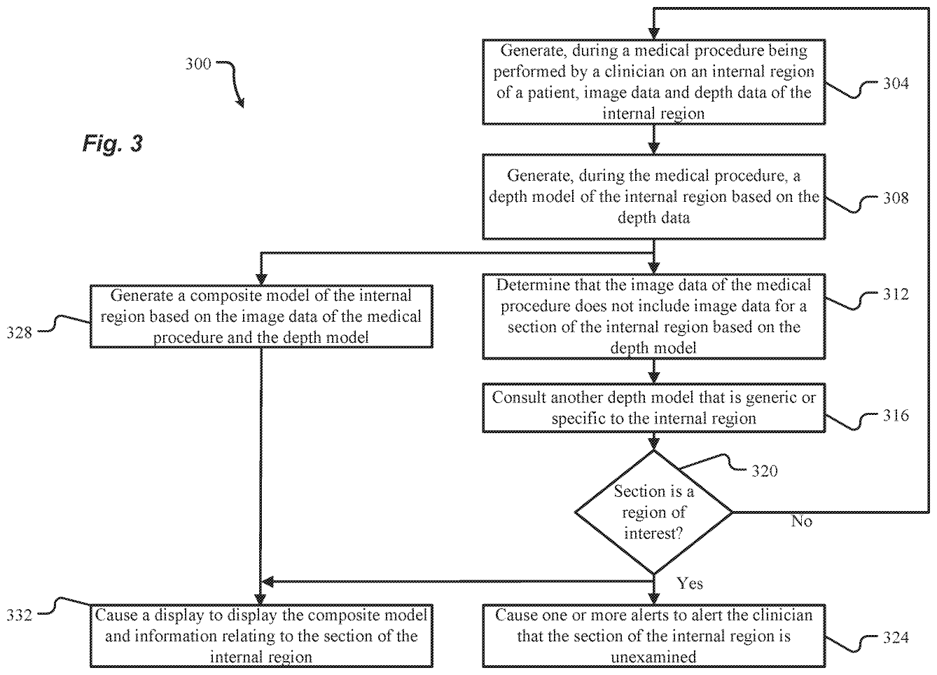

FIG. 3 illustrates a method 300 according to at least one example embodiment. In general, the method 300 may be performed by one or more of the elements from FIG. 1. For example, the method 300 is performed by the processor 116 based on various inputs from other elements of the system 100. However, the method 300 may be performed by additional or alternative elements in the system 100, under control of the processor 116 or another element, for example, as would be recognized by one of ordinary skill in the art.

In operation 304, the method 300 includes generating, during a medical procedure being performed by a clinician on an internal region of a patient, image data and depth data of the internal region. The image data and depth data are generated in accordance with any known technique. For example, as noted above, the image data may include color images and/or video of the medical procedure as captured by camera 136a or camera 136c while the depth data may include depth images and/or video of the medical procedure as captured by camera 136b or camera 136c. In at least one example embodiment, the depth data is derived from the image data, for example, from image data of two cameras 136a in a stereoscopic configuration (or even a single camera 136a). Depth data may be derived from image data in any known manner.

In operation 308, the method 300 includes generating a depth model or depth map of the internal region based on the depth data. For example, the depth model is generated during the medical procedure using the depth data received by the processor 116 from one or more cameras 136. Any known method may be used to generate the depth model. In at least one example embodiment, the depth model is generated in response to a determination that a medical instrument 140 used for the medical procedure enters a general region of interest.

In operation 312, the method 300 includes determining that the image data of the medical procedure does not include image data for a section of the internal region based on the depth model. For example, the processor 116 determines that the image data does not include image data for the section of the internal region when more than a threshold amount of depth data is missing in a region of the depth model. The threshold amount of depth data and a size of the region in the depth model are design parameters based on empirical evidence and/or preference. The region in which depth data is missing may be a unitary region in the depth model. In at least one example embodiment, the region in which depth data is missing may include regions with depth data interspersed among regions without depth data. Parameters of the region (e.g., size, shape, contiguousness) and/or the threshold amount of depth data may be variable and/or selectable during the medical procedure, and, for example, may automatically change depending on a location of the medical instrument 140 within the internal region. For example, as the medical instrument 140 approaches or enters a known region of interest for the internal region, the threshold amount of depth data and/or region parameters may be adjusted to be more sensitive to missing data than in regions generally not of interest. This can further ensure that regions of interest are fully examined while reducing unnecessary alerts and/or processing resources for regions not of interest.

In at least one example embodiment, the clinician may confirm or disconfirm that image data is missing based on a concurrently displayed composite 3D model that includes image data overlaid or mapped to the depth model. For example, the clinician can view whether the composite 3D model includes image data in the region where the system has detected the absence of depth data. Details of the composite 3D model are discussed in more detail below.

In operation 316, the method 300 consults another depth model that may be generic to the internal region of the patient, specific to the internal region of the patient, or both. The another depth model may be a 3D model with or without overlaid image data. For example, in the event that the internal region is an esophagus of the patient, then a generic depth model may be a model of a general esophagus received from a database. The generic model may be modeled on depth and/or image data taken from the internal region(s) (e.g., esophagus) of one or more other patients during other medical procedures. Thus, the generic model may be a close approximation of the depth model generated during the medical procedure that is based on the patient's anatomy. In at least one example embodiment, the generic depth model may include depth data of the internal region of the current patient if, for example, depth and/or image data of the current patient exists from prior medical procedures on the internal region.

In the case where prior medical procedures on the current patient produced depth and/or image data, then the another depth model in operation 316 may be completely specific to the current patient (i.e., not based on data from other patients). In at least one example embodiment, the another depth model includes image and/or depth data specific to the patient as well as generic image and/or depth data. For example, when data specific to the patient exists but is incomplete, then data from a generic model may also be applied to fill the gaps in the patient specific data. The another depth model may be received and/or generated in operation 316 or at some other point within or prior to operations 304 to 312.

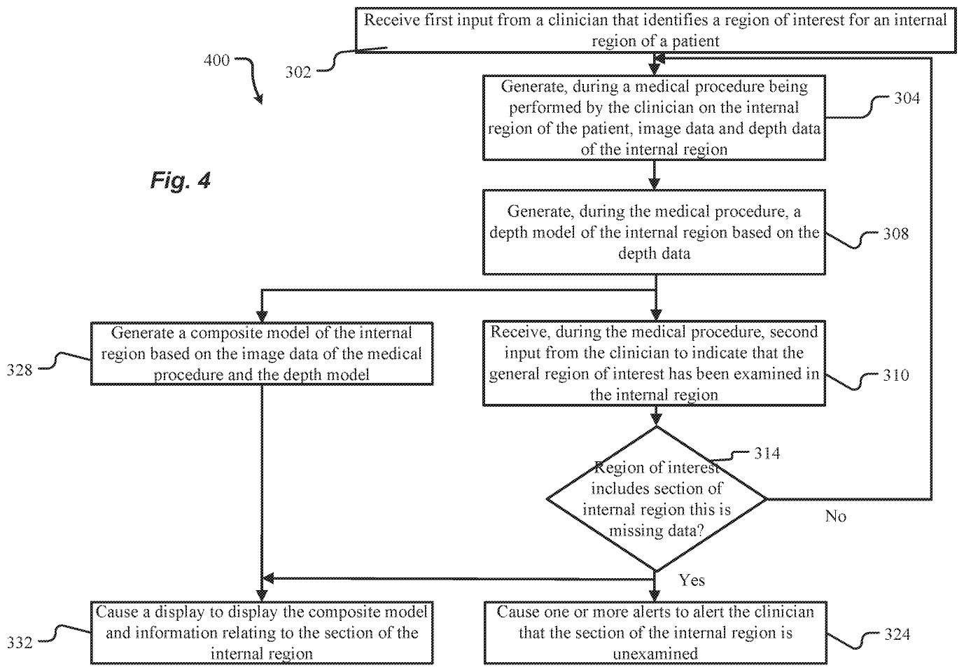

The another depth model consulted in operation 316 may have pre-selected regions of interest to assist with identifying unimaged regions of the internal region during the medical procedure. As discussed in more detail below with reference to FIG. 4, the regions of interest may be selected by the clinician in advance of or during the medical procedure (e.g., using a touch display displaying the another depth model). The regions of interest may be selected with or without the assistance of labeling or direction on the another depth model, where such labeling or direction is generated using the neural network 124 and/or using input from a clinician. For example, using the historical image and/or depth data that generated the another depth model, the neural network 124 can assist with identifying known problem areas (e.g., existing lesions, growths, etc.) and/or or known possible problem areas (e.g., areas where lesions, growths, etc. often appear) by analyzing the historical data and known conclusions drawn therefrom to arrive at one or more other conclusions that could assist the method 300. The regions of interest may be identified by the neural network 124 with or without clinician assistance so as to allow for the method to be completely automated or user controlled.

In operation 320, the method 300 determines whether the section determined in operation 312 to not have image data is a region of interest. For example, the method 300 may determine a location of the medical instrument 140 within the internal region using the additional sensors described above, and compare the determined location to a location of a region of interest from the another depth model. If the location of the medical instrument 140 is within a threshold distance of a region of interest on the another depth model, then the section of the internal region is determined to be a region of interest and the method 300 proceeds to operation 324. If not, then the section of the internal region is determined not to be a region of interest, and the method 300 returns to operation 304 to continue to generate image and depth data. The threshold distance may be a design parameter set based on empirical evidence and/or preference.

The location of the medical instrument 140 may be determined with the assistance of the depth model, the image data, and/or one or more other sensors generally known to help detect location within anatomies. For example, in at least one example embodiment, the depth model, which may not be complete if in the earlier stages of the medical procedure, may be compared to the another depth model. The knowledge of which portions of the depth model are complete versus incomplete compared to the another depth model (which is complete) may be used to estimate a location of the medical instrument 140 in the internal region. For example, the completed portion of the depth model may be overlaid on the another depth model to estimate the location of the medical instrument as the location of the medical instrument 140 as the location where the depth model becomes incomplete compared to the completed another depth model. However, example embodiments are not limited thereto and any known method of determining the location of the medical instrument 140 may be used. Such methods include algorithms for simultaneous localization and mapping (SLAM) techniques that are capable of simultaneously mapping an environment (e.g., the internal region) while tracking a current location within the environment (e.g., a current location of the medical instrument 140). SLAM algorithms may further be assisted by the neural network 124.

In at least one example embodiment, even if the section of the internal region is determined to be not of interest, that section may still be flagged and/or recorded in memory 112 to allow the clinician to revisit the potentially unexamined region at a later time. For example, the system could present the clinician with an audio and/or visual notification that certain sections were determined to be missing image data but determined to be not of interest. The notification may include visual notifications on the depth model and/or on a composite model (that includes the image data overlaid on the depth model) as well as directions for navigating the medical instrument 140 to the sections determined to be missing image data.

Here, it should be appreciated that operations 316 and 320 may be omitted if desired so that the method 300 proceeds from operation 312 directly to operation 324 in order to alert the clinician. Omitting or including operations 316 and 320 may be presented as a choice for the clinician prior to or at any point during the medical procedure.

In operation 324, the method 300 causes one or more alerts to alert the clinician that the section of the internal region is unexamined. The alerts may be audio and/or video in nature. For example, the output device 104 outputs an audio alert, such as a beep or other noise, and/or a visual alert, such as a warning message on a display or warning light.

As shown in FIG. 3, the method 300 may further perform optional operations 328 and 332, for example, in parallel with other operations of FIG. 3.

For example, in operation 328, the method 300 may generate a composite model of the internal region based on the image data of the medical procedure and the depth model. The composite model includes a three-dimensional model of the internal region with the image data of the medical procedure projected onto or overlaid on the depth model. The projection or overlay of the image data onto the depth model may be performed in accordance with known techniques by, for example, aligning the depth model with color images to obtain color information for each point on the depth model.

In operation 332, the method 300 causes a display to display the composite model and information relating to the section of the internal region. The information may include a visualization of the section of the internal region on the composite model. In the event that the section of the internal region on the composite model has been determined to be a region of interest in operation 320, then the information may include audio and/or visual cues and directions for the clinician to navigate the medical instrument 140 to the section of the internal region. The composite model may be interactive on the display. For example, the composite model may be rotatable on x, y, and/or z axes, subject to zoom-in/zoom-out operations, subject to selection of a particular region, and/or subject to other operations generally known to exist for interactive 3D models. The interaction may be performed by the clinician through the input device(s) 128 and/or directly on a touch display.

Here, it should be appreciated that the operations in FIG. 3 may be completely automated. For example, other than guiding the medical instrument 140 or other device with imaging and/or depth camera(s), no user or clinician input is needed throughout operations 304 to 332 if desired. In this case, the another depth model in operation 316 is generated and applied automatically and the region of interest is selected automatically. The automatic generation and application of the another depth model and automatic selection of the region of interest may be assisted by the neural network 124, database 120, processor 116, and/or memory 112.

FIG. 4 illustrates a method 400 according to at least one example embodiment. For example, FIG. 4 illustrates further operations that may be performed additionally or alternatively to the operations shown in FIG. 3 according to at least one example embodiment. Operations depicted in FIG. 4 having the same reference numbers in FIG. 3 are performed in the same manner as described above with reference to FIG. 3. Thus, these operations will not be discussed in detail below. FIG. 4 differs from FIG. 3 in that operations 302, 310, and 314 are included. FIG. 4 relates to an example where the clinician identifies regions of interest for examination and identifies when a region of interest is believed to be examined.

In operation 302, the method 400 receives first input from the clinician that identifies a region of interest in the internal region of the patient. The first input may be input from the clinician on the input device 128 to indicate where the region of interest begins and ends. For example, the clinician may identify a start point and end point or otherwise mark (e.g., encircling) the region of interest on the another depth model discussed in operation 316, where the another depth model is a generic model for the patient, a specific model for the patient, or a combination of both. As noted above, the region of interest may be determined or assisted by the neural network 124, which uses historical data regarding other regions of interest in other medical procedures to conclude that the same regions in the internal region of the patient are also of interest. In this case, the neural network 124 identifies areas on the another depth model that could be of interest and the clinician can confirm or disconfirm that each area is a region of interest with input on the input device 128.

In at least one example embodiment, the first input may identify a region of interest within the internal region of the patient without using the another depth model. In this case, the first input may flag start and end points in the internal region itself using the clinician's general knowledge about the internal region and tracked location of the medical instrument 140 in the internal region. In other words, a start point of the region of interest may be a known or estimated distance from an entry point of the camera(s) 136 into the patient while the end point of the region of interest may be another known or estimated distance from the entry point (or, alternatively, the start point of the region of interest). Tracking the location of the camera(s) 136 within the internal region according to known techniques (e.g., SLAM) enables knowledge of when the camera(s) 136 has entered the start and end points of the region of interest. For example, if the clinician knows that the region of interest starts at 15 cm from the entry point of the camera(s) 136 and ends 30 cm from the entry point, then other sensors on the camera(s) 136 can provide information to the processor 116 to estimate when the camera(s) 136 enter and exit the region of interest. The clinician can trigger start and end points by, for example, a button press on an external control portion of the medical instrument 140.

Although operation 302 is shown as being performed prior to operation 304, operation 302 may be performed at any point prior to operation 310.

The method 400 then performs operations 304 and 308 in accordance with the description of FIG. 3 above to generate image data and depth data and to generate a depth model from the depth data. Operation 302 may also be performed at more than one points prior to operation 310; for example, at a first point, during the medical procedure, to indicate the start of a region of interest and at a second point, during the medical procedure, to indicate the end of a region of interest. Moreover, indications for start and end points of multiple regions of interest can be set.

In operation 310, the method 400 receives, during the medical procedure, second input from the clinician to indicate that the region of interest has been examined in the internal region. The second input may be input on the input device 128 in the same or similar manner as the first input in operation 302. For example, during the medical procedure, the clinician is informed of the region of interest selected in operation 302 through a display of the depth model, the another depth model, and/or the composite model. The clinician provides the second input during the medical procedure when the clinician believes that the region of interest of the internal region has been examined. Operation 310 serves as a trigger to proceed to operation 314.

In operation 314, the method 300 determines, after receiving the second input from the clinician in operation 310, that the region of interest includes the section of the internal region that is missing image data. In other words, operation 344 serves as a double check against the clinician's belief that the entire region of interest has been examined. If, in operation 314, the method 400 determines that the section of the internal region, that is missing data, exists within the region of interest, then the method proceeds to operation 324, which is carried out according to the description of FIG. 3. If not, the method 400 proceeds back to operation 304 to continue to generate image data and depth data of the internal region. In the case that the method 400 proceeds to operation 324, the one or more alerts include an alert to inform the clinician that at least a portion of the region of interest was left unexamined.

Operation 314 may be carried out in a same or similar manner as operation 312 in FIG. 3. For example, in order to determine whether the region of interest includes a section of the internal region that is missing image data, the method 400 evaluates whether more than a threshold amount of depth data is missing in the depth model generated in operation 308, where the missing depth data is in a region that corresponds to part of the region of interest. As in the method of FIG. 3, the method 400 includes mapping the region of interest selected in operation 302 onto the depth model generated in operation 308 according to known techniques.

Here, it should be appreciated that the method 400 provides the clinician or other user the ability to provide input for selecting a region of interest and/or for double checking the clinician's belief that the region of interest has been fully examined.

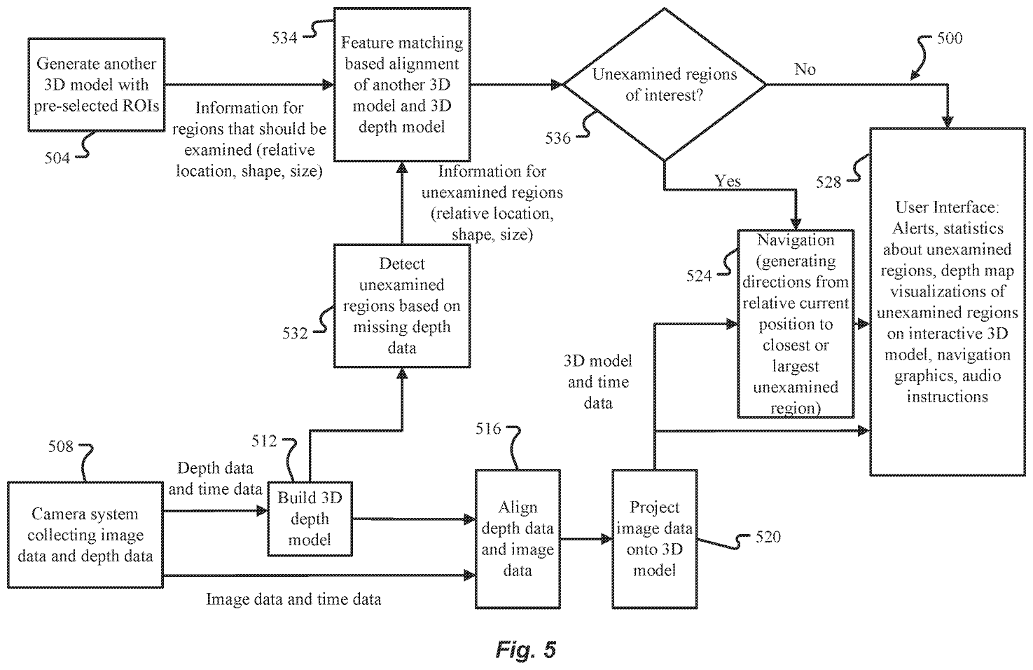

FIG. 5 illustrates a workflow 500 for a medical procedure according to at least one example embodiment. The operations of FIG. 5 are described with reference to FIGS. 1-4 and illustrate how the elements and operations in FIGS. 1-4 fit within a workflow of a medical procedure on patient. Although the operations in FIG. 5 are described in numerical order, it should be appreciated that one or more of the operations may occur at a different point in time than shown and/or may occur simultaneously with other operations. As in FIGS. 3 and 4, the operations in FIG. 5 may be carried out by one or more of the elements in the system 100.

In operation 504, the workflow 500 includes generating another model, for example, a 3D depth model with pre-selected regions of interest (see operations 302 and 316, for example). Operation 504 may include generating information on a relative location, shape, and/or size of a region of interest and passing that information to operation 534, discussed in more detail below.

In operation 508, a camera system (e.g., cameras 136a and 136b) collects image data and depth data of a medical procedure being performed by a clinician in accordance with the discussion of FIGS. 1-4.

In operation 512, depth and time data are used to build a 3D depth model, while in operation 516, image data and time data are used along with the depth model to align the depth data with the image data. For example, the time data for each of the image data and depth data may include time stamps for each frame or still image taken with the camera(s) 136 so that in operation 516, the processor 116 can match time stamps of the image data to time stamps of the depth data, thereby ensuring that the image data and the depth data are aligned with one another at each instant in time.

In operation 520, the image data is projected onto the depth model to form a composite model as a 3D color image model of the internal region. The 3D composite model and time data are used to assist with navigation of the camera(s) 136 and/or medical instrument 140 in operation 524, and may be displayed on a user interface of a display in operation 528.

In operation 524, the workflow 500 performs navigation operations, which may include generating directions from a current position of the camera(s) 136 to a closest and/or largest unexamined region. The directions may be produced as audio and/or visual directions on the user interface in operation 528. Example audio directions include audible “left, right, up, down” directions while example video directions include visual left, right, up, down arrows on the user interface. Lengths and/or colors of the arrows may change as the clinician navigates toward an unexamined region. For example, an arrow may become shorter and/or change colors as the camera(s) 136 get closer to the unexamined region.

In operation 528, a user interface displays or generates various information about the medical procedure. For example, the user interface may include alerts that a region is unexamined, statistics about unexamined regions (e.g., how likely the unexamined region contains something of interest), visualizations of the unexamined regions, an interactive 3D model of the internal region, navigation graphics, audio instructions, and/or any other information that may be pertinent to the medical procedure and potentially useful to the clinician.

Operation 532 includes receiving the depth model from operation 512 and detecting one or more unexamined regions of the internal region based on depth data missing from the depth model, for example, as in operation 312 described above.

Operation 534 includes receiving information regarding the unexamined regions, for example, information regarding a relative location, a shape, and/or size of the unexamined regions. Operation 534 further includes using this information to perform feature matching with the depth model from operation 512 and the another model from operation 534. The feature matching between models may be performed according to any known technique, which may utilize mesh modeling concepts, point cloud concepts, scale-invariant feature transform (SIFT) concepts, and/or the like.

The workflow 500 then moves to operation 536 to determine whether the unexamined regions are of interest based on the feature matching in operation 534. This determination may be performed in accordance with, for example, operation 320 described above. Information regarding any unexamined regions and whether they are of interest is passed to operations 524 and 528. For example, if an unexamined region is of interest, then that information is used in operation 524 to generate information that directs the clinician from a current position to a closest largest unexamined region. The directions generated in operation 524 may be displayed on the user interface in operation 528. Additionally or alternatively, if an unexamined region is determined to not be of interest, then a notification of the same may be sent to the user interface along with information regarding a location of the unexamined region not of interest. This enables the clinician to double check whether the region is actually not of interest. The clinician can then indicate that the region is of interest and directions to the region can be generated as in operation 524.

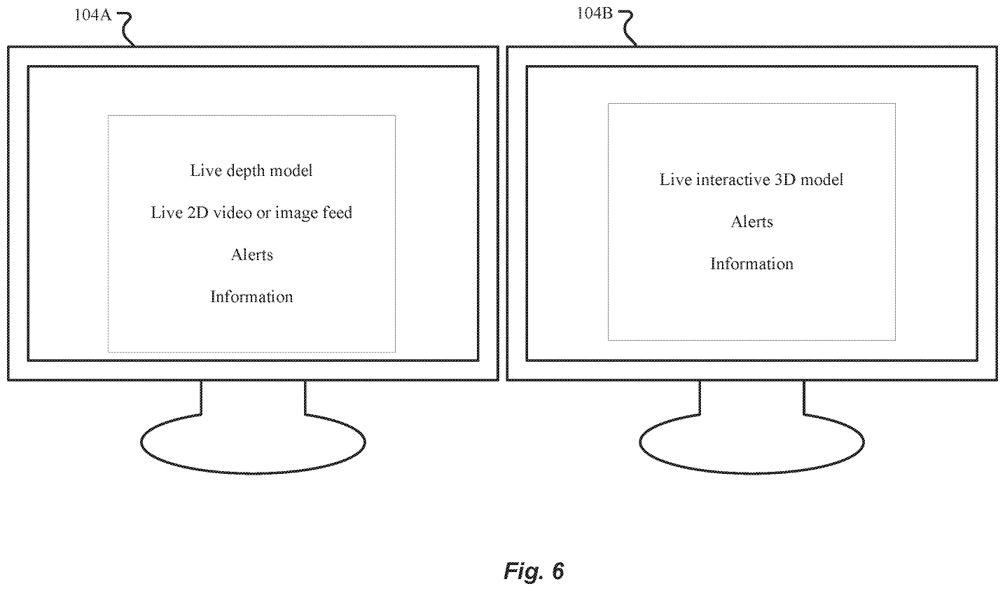

FIG. 6 illustrates example output devices 104A and 104B as displays, for example, flat panel displays. Although two output devices are shown, more or fewer output devices may be included if desired.

In at least one example embodiment, output device 104A displays a live depth model of the current medical procedure. A variety of functions may be available to interact with the depth model, which may include zoom functions (in and out), rotate functions (x, y, and/or z axis rotation), region selection functions, and/or the like. Output device 104A may further display the live 2D video or still image feed of the internal region from a camera 136a. The output device 104A may further display one or more alerts, for example, alerts regarding missing data in the live depth model, alerts that a region is unexamined, and the like. The output device 104A may further display various information, such as graphics for navigating the medical instrument 140 to an unexamined region, statistics about the medical procedure and/or unexamined region, and the like.

Output device 104B may display an interactive composite 3D model with the image data overlaid or projected onto the depth model. A variety of functions may be available to interact with the composite 3D model, which may include zoom functions (in and out), rotate functions (x, y, and/or z axis rotation), region selection functions, and/or the like. Similar to the output device 104A, the output device 104B may display alerts and/or other information about the medical procedure. Displaying the live depth and image feeds as well as the 3D composite model during the medical procedure may help ensure that all regions are examined.

The output devices 104A and/or 104B may further display a real-time location of the medical instrument 140 and/or other device with camera(s) 136 within the depth model and/or the composite model. In the event of detecting an unexamined region, the aforementioned navigation arrows may be displayed on the model, and may vary in color, speed at which they may flash, and/or length according to how near or far the camera is to an unexamined region.

Here, it should be appreciated that the operations in FIGS. 3-5 do not necessarily have to be performed in the order shown and described. One skilled in the art should appreciate that other operations within FIGS. 3-5 may be reordered according to design preferences.

Although example embodiments have been described with respect to medical procedures that occur internal to a patient, example embodiments may also be applied to non-medical procedures of internal regions that are camera assisted (e.g., examination of pipes or other structures that are difficult to examine from an external point of view).

In view of foregoing description, it should be appreciated that example embodiments provide efficient methods for automatically identifying potentially unexamined regions of an anatomy and providing appropriate alerts and/or instructions to guide a clinician user to the unexamined regions, thereby ensuring that all regions that all intended regions are examined.

At least one example embodiment is directed to a device including a memory including instructions, and a processor that executes the instructions to generate, during a medical procedure being performed by a clinician on an internal region of a patient, image data and depth data for the internal region, generate, during the medical procedure, a depth model of the internal region of the patient based on the depth data, determine that the image data of the medical procedure does not include image data for a section of the internal region based on the depth model, and cause one or more alerts to alert the clinician that the section of the internal region is unexamined.

According to at least one example embodiment, the instructions include instructions that cause the processor to generate a composite model of the internal region based on the image data of the medical procedure and the depth model, and cause a display to display the composite model and information relating to the section of the internal region.

According to at least one example embodiment, the composite model includes a three-dimensional model of the internal region with the image data of the medical procedure projected onto the depth model.

According to at least one example embodiment, the one or more alerts include an alert displayed on the display.

According to at least one example embodiment, the information includes a visualization of the section of the internal region on the composite model.

According to at least one example embodiment, the information includes visual and/or audio clues and directions for the clinician to navigate a medical instrument to the section of the internal region.

According to at least one example embodiment, the instructions include instructions that cause the processor to determine that the section of the internal region is a region of interest based on another depth model that is generic to or specific to the internal region. The one or more alerts include an alert to inform the clinician that the section of the internal region should be examined.

According to at least one example embodiment, the instructions include instructions that cause the processor to receive first input from the clinician that identifies a region of interest in the internal region of the patient, and receive, during the medical procedure, second input from the clinician to indicate that the region of interest has been examined.

According to at least one example embodiment, the instructions include instructions that cause the processor to determine, after receiving the second input from the clinician, that the region of interest includes the section of the internal region that is missing data. The one or more alerts include an alert to inform the clinician that the at least a portion of the region of interest was left unexamined.

According to at least one example embodiment, the processor generates the depth model in response to a determination that a medical instrument used for the medical procedure enters the region of interest.

According to at least one example embodiment, the processor determines that the image data does not include image data for the section of the internal region when more than a threshold amount of depth data is missing in a region of the depth model.

According to at least one example embodiment, the instructions include instructions to cause the processor to execute a first machine learning algorithm to determine a region of interest within the internal region and to determine a path for navigating a medical instrument to the region of interest, and execute a second machine learning algorithm to cause a robotic device to navigate the medical instrument to the region of interest within the internal region.

At least one example embodiment is directed to a system including a display, a medical instrument, and a device. The device includes a memory including instructions and a processor that executes the instructions to generate, during a medical procedure being performed by a clinician on an internal region of a patient, image data and depth data for the internal region, generate, during the medical procedure, a depth model of the internal region of the patient based on the depth data, determine that the image data of the medical procedure does not include image data for a section of the internal region based on the depth model, cause/generate one or more alerts to alert the clinician that the section of the internal region is unexamined.

According to at least one example embodiment, the medical instrument includes a stereoscopic camera that provides the image data. The depth data is derived from the image data.

According to at least one example embodiment, the medical instrument includes a depth sensor that provides the depth data, and an image sensor to provide the image data. The depth sensor and the image sensor are arranged on the medical instrument to have overlapping fields of view.

According to at least one example embodiment, the medical instrument includes a sensor including depth pixels that provide the depth data and imaging pixels that provide the image data.

According to at least one example embodiment, the system includes a robotic device for navigating the medical instrument within the internal region, and the instructions include instructions that cause the processor to execute a first machine learning algorithm to determine a region of interest within the internal region and to determine a path for navigating the medical instrument to the region of interest, and execute a second machine learning algorithm to cause the robotic device to navigate the medical instrument to the region of interest within the internal region.

According to at least one example embodiment, the system includes an input device that receives input from the clinician to approve the path for navigating the medical instrument to the region of interest before the processor executes the second machine learning algorithm.

At least one example embodiment is directed to a method including generating, during a medical procedure being performed by a clinician on an internal region of a patient, image data and depth data for the internal region, generating, during the medical procedure, a depth model of the internal region of the patient based on the depth data, determining that the image data does not include image data for a section of the internal region based on the depth model and causing one or more alerts to alert the clinician that the section of the internal region is unexamined.

According to at least one example embodiment, the method includes generating an interactive three-dimensional model of the internal region with the image data of the medical procedure projected onto the depth model, and causing the display to display the interactive three-dimensional model and visual and/or audio cues and directions to direct a clinician performing the medical procedure to the section of the internal region.

Any one or more of the aspects/embodiments as substantially disclosed herein.

Any one or more of the aspects/embodiments as substantially disclosed herein optionally in combination with any one or more other aspects/embodiments as substantially disclosed herein.

One or more means adapted to perform any one or more of the above aspects/embodiments as substantially disclosed herein.

The phrases “at least one,” “one or more,” “or,” and “and/or” are open-ended expressions that are both conjunctive and disjunctive in operation. For example, each of the expressions “at least one of A, B and C,” “at least one of A, B, or C,” “one or more of A, B, and C,” “one or more of A, B, or C,” “A, B, and/or C,” and “A, B, or C” means A alone, B alone, C alone, A and B together, A and C together, B and C together, or A, B and C together.

The term “a” or “an” entity refers to one or more of that entity. As such, the terms “a” (or “an”), “one or more,” and “at least one” can be used interchangeably herein. It is also to be noted that the terms “comprising,” “including,” and “having” can be used interchangeably.

Aspects of the present disclosure may take the form of an embodiment that is entirely hardware, an embodiment that is entirely software (including firmware, resident software, micro-code, etc.) or an embodiment combining software and hardware aspects that may all generally be referred to herein as a “circuit,” “module,” or “system.” Any combination of one or more computer-readable medium(s) may be utilized. The computer-readable medium may be a computer-readable signal medium or a computer-readable storage medium.

A computer-readable storage medium may be, for example, but not limited to, an electronic, magnetic, optical, electromagnetic, infrared, or semiconductor system, apparatus, or device, or any suitable combination of the foregoing. More specific examples (a non-exhaustive list) of the computer-readable storage medium would include the following: an electrical connection having one or more wires, a portable computer diskette, a hard disk, a random access memory (RAM), a read-only memory (ROM), an erasable programmable read-only memory (EPROM or Flash memory), an optical fiber, a portable compact disc read-only memory (CD-ROM), an optical storage device, a magnetic storage device, or any suitable combination of the foregoing. In the context of this document, a computer-readable storage medium may be any tangible medium that can contain or store a program for use by or in connection with an instruction execution system, apparatus, or device.

The terms “determine,” “calculate,” “compute,” and variations thereof, as used herein, are used interchangeably and include any type of methodology, process, mathematical operation or technique.

Example embodiments may be configured according to the following:

(1) A device comprising:

a memory including instructions; and

a processor that executes the instructions to:
generate, during a medical procedure being performed by a clinician on an internal region of a patient, image data and depth data for the internal region;
generate, during the medical procedure, a depth model of the internal region of the patient based on the depth data;
determine that the image data of the medical procedure does not include image data for a section of the internal region based on the depth model; and
cause one or more alerts to alert the clinician that the section of the internal region is unexamined.
   (2) The device of (1), wherein the instructions include instructions that cause the processor to:

generate a composite model of the internal region based on the image data of the medical procedure and the depth model; and

cause a display to display the composite model and information relating to the section of the internal region.

(3) The device of one or more of (1) to (2), wherein the composite model includes a three-dimensional model of the internal region with the image data of the medical procedure projected onto the depth model.
(4) The device of one or more of (1) to (3), wherein the one or more alerts include an alert displayed on the display.
(5) The device of one or more of (1) to (4), wherein the information includes a visualization of the section of the internal region on the composite model.
(6) The device of one or more of (1) to (5), wherein the information includes visual and/or audio cues and directions for the clinician to navigate a medical instrument to the section of the internal region.
(7) The device of one or more of (1) to (6), wherein the instructions include instructions that cause the processor to:

determine that the section of the internal region is a region of interest based on another depth model that is generic to or specific to the internal region, wherein the one or more alerts include an alert to inform the clinician that the section of the internal region should be examined.

(8) The device of one or more of (1) to (7), wherein the instructions include instructions that cause the processor to:

receive first input from the clinician that identifies a region of interest in the internal region of the patient; and

receive, during the medical procedure, second input from the clinician to indicate that the region of interest has been examined.

(9) The device of one or more of (1) to (8), wherein the instructions include instructions that cause the processor to:

determine, after receiving the second input from the clinician, that the region of interest includes the section of the internal region, wherein the one or more alerts include an alert to inform the clinician that the at least a portion of the region of interest was left unexamined.

(10) The device of one or more of (1) to (9), wherein the processor generates the depth model in response to a determination that a medical instrument used for the medical procedure enters the region of interest.
(11) The device of one or more of (1) to (10), wherein the processor determines that the image data does not include image data for the section of the internal region when more than a threshold amount of depth data is missing in a region of the depth model.
(12) The device of one or more of (1) to (11), wherein the instructions include instructions to cause the processor to:

execute a first machine learning algorithm to determine a region of interest within the internal region and to determine a path for navigating a medical instrument to the region of interest; and

execute a second machine learning algorithm to cause a robotic device to navigate the medical instrument to the region of interest within the internal region.

(13) A system, comprising:

a display;

a medical instrument; and

a device including:
a memory including instructions; and
a processor that executes the instructions to:
   generate, during a medical procedure being performed by a clinician on an internal region of a patient, image data and depth data for the internal region;
   generate, during the medical procedure, a depth model of the internal region of the patient based on the depth data;
   determine that the image data of the medical procedure does not include image data for a section of the internal region based on the depth model; and
   cause one or more alerts to alert the clinician that the section of the internal region is unexamined.
      (14) The system of one or more of (13), wherein the medical instrument includes a stereoscopic camera that provides the image data, and wherein the depth data is derived from the image data.
      (15) The system of one or more of (13) to (14), wherein the medical instrument includes a depth sensor that provides the depth data, and an image sensor to provide the image data, and wherein depth sensor and the image sensor are arranged on the medical instrument to have overlapping fields of view.
      (16) The system of one or more of (13) to (15), wherein the medical instrument includes a sensor including depth pixels that provide the depth data and imaging pixels that provide the image data.
      (17) The system of one or more of (13) to (16), further comprising:

a robotic device for navigating the medical instrument within the internal region, wherein the instructions include instructions that cause the processor to:
execute a first machine learning algorithm to determine a region of interest, or a set of regions of interest, within the internal region and to determine a path for navigating to the region(s) of interest; and
execute a second machine learning algorithm to cause the robotic device to navigate to the region(s) of interest within the internal region.
   (18) The system of one or more of (13) to (17), further comprising:

an input device that receives input from the clinician to approve the path for navigating to the region of interest before the processor executes the second machine learning algorithm.

(19) A method comprising:

generating, during a medical procedure being performed by a clinician on an internal region of a patient, image data and depth data for the internal region;

generating, during the medical procedure, a depth model of the internal region of the patient based on the depth data;

determining that the image data does not include image data for a section of the internal region based on the depth model; and

causing one or more alerts to alert the clinician that the section of the internal region is unexamined.

(20) The method of (19), further comprising:

generating an interactive three-dimensional model of the internal region with the image data of the medical procedure projected onto the depth model; and

causing the display to display the interactive three-dimensional model and visual and/or audio cues and directions to direct a clinician performing the medical procedure to the section of the internal region.

What is claimed is:

1. A device comprising:
a memory including instructions; and
a processor that executes the instructions to:
generate, during a medical procedure being performed by a clinician on an internal region of a patient, image data and depth data for the internal region;
generate, during the medical procedure, a depth model of the internal region of the patient based on the depth data;
determine that the image data of the medical procedure does not include image data for a section of the internal region based on the depth model; and
cause one or more alerts to alert the clinician that the section of the internal region is unexamined.

2. The device of claim 1, wherein the instructions include instructions that cause the processor to:
generate a composite model of the internal region based on the image data of the medical procedure and the depth model; and
cause a display to display the composite model and information relating to the section of the internal region.

3. The device of claim 2, wherein the composite model includes a three-dimensional model of the internal region with the image data of the medical procedure projected onto the depth model.

4. The device of claim 2, wherein the one or more alerts include an alert displayed on the display.

5. The device of claim 2, wherein the information includes a visualization of the section of the internal region on the composite model.

6. The device of claim 2, wherein the information includes visual and/or audio cues and directions for the clinician to navigate a medical instrument to the section of the internal region.

7. The device of claim 2, wherein the instructions include instructions that cause the processor to:
determine that the section of the internal region is a region of interest based on another depth model that is generic to or specific to the internal region, wherein the one or more alerts include an alert to inform the clinician that the section of the internal region was left unexamined and should be examined.

8. The device of claim 2, wherein the instructions include instructions that cause the processor to:
receive first input from the clinician that identifies a region of interest in the internal region of the patient; and
receive, during the medical procedure, second input from the clinician to indicate that the region of interest has been examined.

9. The device of claim 8, wherein the instructions include instructions that cause the processor to:
determine, after receiving the second input from the clinician, that the region of interest includes the section of the internal region, wherein the one or more alerts include an alert to inform the clinician that the at least a portion of the region of interest was left unexamined.

10. The device of claim 8, wherein the processor generates the depth model in response to a determination that a medical instrument used for the medical procedure enters the region of interest.

11. The device of claim 1, wherein the processor determines that the image data does not include image data for the section of the internal region when more than a threshold amount of depth data is missing in a region of the depth model.

12. The device of claim 1, wherein the instructions include instructions to cause the processor to:
execute a first machine learning algorithm to determine one or more regions of interest within the internal region and to determine a path for navigating a medical instrument to the one or more regions of interest; and
execute a second machine learning algorithm to cause a robotic device to navigate the medical instrument to the one or more regions of interest within the internal region.

13. A system, comprising:
a display;
a medical instrument; and
a device including:
a memory including instructions; and
a processor that executes the instructions to:
generate, during a medical procedure being performed by a clinician on an internal region of a patient, image data and depth data for the internal region;
generate, during the medical procedure, a depth model of the internal region of the patient based on the depth data;
determine that the image data of the medical procedure does not include image data for a section of the internal region based on the depth model; and
cause one or more alerts to alert the clinician that the section of the internal region is unexamined.

14. The system of claim 13, wherein the medical instrument includes a stereoscopic camera that provides the image data, and wherein the depth data is derived from the image data.

15. The system of claim 13, wherein the medical instrument includes a depth sensor that provides the depth data, and an image sensor to provide the image data, and wherein depth sensor and the image sensor are arranged on the medical instrument to have overlapping fields of view.

16. The system of claim 13, wherein the medical instrument includes a sensor including depth pixels that provide the depth data and imaging pixels that provide the image data.

17. The system of claim 16, further comprising:
a robotic device for navigating the medical instrument within the internal region, wherein the instructions include instructions that cause the processor to:
execute a first machine learning algorithm to determine one or more regions of interest within the internal region and to determine a path for navigating the medical instrument to the one or more regions of interest; and
execute a second machine learning algorithm to cause the robotic device to navigate the medical instrument to the one or more regions of interest within the internal region.

18. The system of claim 17, further comprising:
an input device that receives input from the clinician to approve the path for navigating to the one or more regions of interest before the processor executes the second machine learning algorithm.

19. A method comprising:
generating, during a medical procedure being performed by a clinician on an internal region of a patient, image data and depth data for the internal region;
generating, during the medical procedure, a depth model of the internal region of the patient based on the depth data;
determining that the image data does not include image data for a section of the internal region based on the depth model; and
causing one or more alerts to alert the clinician that the section of the internal region is unexamined.

20. The method of claim 19, further comprising:
generating an interactive three-dimensional model of the internal region with the image data of the medical procedure projected onto the depth model; and
causing the display to display the interactive three-dimensional model and visual cues and directions to direct a clinician performing the medical procedure to the section of the internal region.
